(19) United States
(12) Patent Application Publication
(10) Pub. No.: US 2013/0071855 A1
(43) Pub. Date: Mar. 21, 2013

(54) FLC AS BIOMARKER

(71) Applicants: Arthur Randell Bradwell, Birmingham (GB); Richard Hughes, Birmingham (GB)

(72) Inventors: Arthur Randell Bradwell, Birmingham (GB); Richard Hughes, Birmingham (GB)

(73) Assignee: THE BINDING SITE GROUP LIMITED, Birmingham (GB)

(21) Appl. No.: 13/635,160

(22) PCT Filed: Mar. 16, 2011
(86) PCT No.: PCT/GB11/50518

§ 371 Date: Nov. 28, 2012

(30) Foreign Application Priority Data
Mar. 17, 2010 (GB) 1004442.8

(51) Int. Cl. G01N 33/566; G01N 33/573; G01N 21/82

(52) U.S. Cl. 435/7.4; 436/501; 435/7.92

(57) ABSTRACT

The invention provides a method of identifying a subject likely to have liver disease, or for determining the prognosis of a subject previously identified as having a liver disease comprising detecting an amount of free light chains in a sample from the subject, wherein a higher amount of FLC is associated with an increased likelihood of the subject having a liver disease or an increased likelihood of having a poor prognosis of a liver disease. Assay kits for use in such methods are also provided.

The invention relates to a novel biomarker for patients with liver disease, identifying subjects with more serious disease and a worse prognosis.

Liver disease is a serious condition which, when left untreated, may eventually lead to the requirement of liver transplant thus an accessible procedure to identify and assess the degree of liver disease is beneficial. Abnormal B-cell activation is known in autoimmune liver diseases, as illustrated by the emergence of autoantibodies and high levels of immunoglobulins in many chronic liver diseases. It was considered that sFLC concentrations may be elevated in liver disease and thus provide a pathological indication of the disease process.

The Applicants have for many years studied free light chains as a way of assaying for a wide-range of monoclonal gammopathies in patients. The use of such free light chains in diagnosis is reviewed in detail in the book “Serum Free Light Chain Analysis, Fifth Edition (2008) A. R. Bradwell et al, ISBN 0704427028”.

Antibodies comprise heavy chains and light chains. They usually have a two-fold symmetry and are composed of two identical heavy chains and two identical light chains, each containing variable and constant region domains. The variable domains of each light-chain/heavy-chain pair combine to form an antigen-binding site, so that both chains contribute to the antigen-binding specificity of the antibody molecule. Light chains are of two types, κ and λ and any given antibody molecule is produced with either light chain but never both. There are approximately twice as many κ as λ molecules produced in humans, but this is different in some mammals. Usually the light chains are attached to heavy chains. However, some unattached “free light chains” are detectable in the serum or urine of individuals. Free light chains may be specifically identified by raising antibodies against the surface of the free light chain that is normally hidden by the binding of the light chain to the heavy chain. In free light chains (FLC) this surface is exposed, allowing it to be detected immunologically. Commercially available kits for the detection of κ or λ free light chains include, for example, “Freelite™”, manufactured by The Binding Site Limited, Birmingham, United Kingdom. The Applicants have previously identified that determining the amount of free κ/free λ ratios, aids the diagnosis of monoclonal gammopathies in patients. It has been used, for example, as an aid in the diagnosis of intact immunoglobulin multiple myeloma (MM), light chain MM, non-secretory MM, AL amyloidosis, light chain deposition disease, smouldering MM, plasmacytoma and MGUS (monoclonal gammopathies of undetermined significance). Detection of FLC has also been used, for example, as an aid to the diagnosis of other B-cell dyscrasia and indeed as an alternative to urinary Bence Jones protein analysis for the diagnosis of monoclonal gammopathies in general.

Conventionally, an increase in one of the λ or κ light chains and a consequently abnormal ratio is looked for. For example, multiple myelomas result from the monoclonal multiplication of a malignant plasma cell, resulting in an increase in a single type of cell producing a single type of immunoglobulin. This results in an increase in the amount of free light chain, either λ or κ, observed within an individual. This increase in concentration may be determined, and usually the ratio of the free κ to free λ is determined and compared with the normal range. This aids in the diagnosis of monoclonal disease. Moreover, the free light chain assays may also be used for the following of treatment of the disease in patients. Prognosis of, for example, patients after treatment for AL amyloidosis may be carried out.

Katzmann et al (Clin. Chem. (2002); 48(9): 1437-1944) discuss serum reference intervals and diagnostic ranges for free κ and free λ immunoglobulins in the diagnosis of monoclonal gammopathies. Individuals from 21-90 years of age were studied by immunoassay and compared to results obtained by immunofixation to optimise the immunoassay for the detection of monoclonal free light chains (FLC) in individuals with B-cell dyscrasia.

The amount of κ and λ FLC and the κ/λ ratios were recorded allowing a reference interval to be determined for the detection of B-cell dyscrasias.

The concentration of FLC in serum from individuals that are apparently healthy is influenced by the ability of the individual's kidneys to filter and excrete FLC. In individuals where FLC clearance is restricted, there is an increase in the levels of FLC found in serum. As a consequence, it is now believed that FLC is a good marker of renal function. Because monomeric FLC kappa molecules (25 kDa) are of different size to dimeric lambda molecules (50 kDa), together they are better markers of glomerular filtration than, for example, creatinine 113 kDa). However, in contrast to creatinine, production of FLCs may result as a consequence of many diseases, so serum FLCs will typically not be used as a renal function marker, in isolation.

However, markers of B-cell proliferation/activity are important and because B-cells are responsible for making FLCs, this is clinically useful. FLC production is an early indicator of B-cell up-regulation. In this respect it can complement the use of CRP which is a T-cell mediated marker of inflammatory responses.

High FLC concentrations may well be an indication of chronic renal disorders or chronic B-cell activation due to disease pathology or B-cell dyscrasias. Hence, an abnormal FLC assay result may be a marker of a variety of disorders that currently require several tests in combination. The converse of this, when the FLC assay results are normal, indicates good renal function, no inflammatory conditions and no evidence of B-cell dyscrasia.

The applicant studied samples from a number of patients having different liver diseases. The specific liver disease was compared to the FLC concentration. FLC concentrations were shown to be significantly elevated above published FLC levels. Following correction for renal function FLC values remained elevated above levels for a healthy population following their correction for renal function.

The invention provides a method of identifying a subject likely to have liver disease, or for determining the prognosis of a subject previously identified as having a liver disease comprising detecting an amount of free light chains in a sample from the subject, wherein a higher amount of FLC is associated with an increased likelihood of the subject having a liver disease or an increased likelihood of having a poor prognosis of a liver disease. That is, a higher level of FLC indicates that the subject has a liver disease or that the liver disease has progressed further than a lower level of FLC.

The method may also be used to follow the treatment of such a disease; for example, a lower level of FLC after treatment indicates that the treatment is successful.

Liver diseases are characterised by an excess death rate of liver cells, such as hepatocytes. The underlying cause of liver disease may be related to viral infections such as hepatitis, autoimmune conditions such as autoimmune hepatitis, substance abuse such as alcoholic liver disease or cryptogenic.

The liver disease may be a liver disease including alcohol related liver disease, autoimmune hepatitis, autoimmune sclerosing cholangitis, non-alcoholic fatty liver disease, primary biliary cirrhosis (PBC), cryptogenic cirrhosis, granulomatous hepatitis, and non alcoholic steato-hepatitis

Typically a normal value above which the FLC level is considered to be significant is 19.4 mg/L for kappa FLC, 26.3 mg/L for lambda, and 45.7 mg/L for total FLC.

The FLC value may be corrected for renal function by for example dividing FLC levels by cystatin C values.

The FLC may be kappa or lambda FLC. However, preferably the total FLC concentration is measured, as detecting kappa FLC or lambda FLC alone may miss, for example abnormally high levels of one or other FLC produced for example monoclonally in the patient.

Total free light chain means the total amount of free kappa plus free lambda light chains in a sample.

Preferably the subject does not necessarily have symptoms of a B-cell associated disease. The symptoms may include recurrent infections, bone pain and fatigue. Such a B-cell associated disease is preferably not a myeloma, (such as intact immunoglobulin myeloma, light chain myeloma, non-secretory myeloma), an MGUS, AL amyloidosis, Waldenstrom's macroglobulinaemia, Hodgkin's lymphoma, follicular centre cell lymphoma, chronic lymphocytic leukaemia, mantle cell lymphoma, pre-B cell leukaemia or acute lymphoblastic leukaemia. Moreover, the individual typically does not have reduced bone marrow function. The individual typically does not have an abnormal κ:λ FLC ratio, typically found in many such diseases.

The term “total free light chains” means the amount of κ and λ free light chains in the sample from the subject.

The sample is typically a sample of serum from the subject. However, whole blood, plasma, urine or other samples of tissue or fluids may also potentially be utilised.

Typically the FLC, such as total FLC, is determined by immunoassay, such as ELISA assays or utilising fluorescently labeled beads, such as Luminex™ beads.

Sandwich assays, for example, use antibodies to detect specific antigens. One or more of the antibodies used in the assay may be labeled with an enzyme capable of converting a substrate into a detectable analyte. Such enzymes include horseradish peroxidase, alkaline phosphatase and other enzymes known in the art. Alternatively, other detectable tags or labels may be used instead of, or together with, the enzymes. These include radioisotopes, a wide range of coloured and fluorescent labels known in the art, including fluorescein, Alexa fluor, Oregon Green, BODIPY, rhodamine red, Cascade Blue, Marina Blue, Pacific Blue, Cascade Yellow, gold; and conjugates such as biotin (available from, for example, Invitrogen Ltd, United Kingdom). Dye sols, chemiluminescent labels, metallic sols or coloured latex may also be used. One or more of these labels may be used in the ELISA assays according to the various inventions described herein or alternatively in the other assays, labeled antibodies or kits described herein.

The construction of sandwich-type assays is itself well known in the art. For example, a “capture antibody” specific for the FLC is immobilised on a substrate. The “capture antibody” may be immobilised onto the substrate by methods which are well known in the art. FLC in the sample are bound by the “capture antibody” which binds the FLC to the substrate via the “capture antibody”.

Unbound immunoglobulins may be washed away.

In ELISA or sandwich assays the presence of bound immunoglobulins may be determined by using a labeled “detecting antibody” specific to a different part of the FLC of interest than the binding antibody.

Flow cytometry may be used to detect the binding of the FLC of interest. This technique is well known in the art for, e.g. cell sorting. However, it can also be used to detect labeled particles, such as beads, and to measure their size. Numerous text books describe flow cytometry, such as Practical Flow Cytometry, 3rd Ed. (1994), H. Shapiro, Alan R. Liss, New York, and Flow Cytometry, First Principles (2nd Ed.) 2001, A. L. Given, Wiley Liss.

One of the binding antibodies, such as the antibody specific for FLC, is bound to a bead, such as a polystyrene or latex bead. The beads are mixed with the sample and the second detecting antibody. The detecting antibody is preferably labeled with a detectable label, which binds the FLC to be detected in the sample. This results in a labeled bead when the FLC to be assayed is present.

Other antibodies specific for other analytes described herein may also be used to allow the detection of those analytes.

Labeled beads may then be detected via flow cytometry. Different labels, such as different fluorescent labels may be used for, for example, the anti-free λ and anti-free κ antibodies. Other assays such as generally known liver function tests may be used in combination with this method.

Alternatively, or additionally, different sized beads may be used for different antibodies, for example for different marker specific antibodies. Flow cytometry can distinguish between different sized beads and hence can rapidly determine the amount of each FLC or other analyte in a sample.

An alternative method uses the antibodies bound to, for example, fluorescently labeled beads such as commercially available Luminex™ beads. Different beads are used with different antibodies. Different beads are labeled with different fluorophore mixtures, thus allowing different analytes to be determined by the fluorescent wavelength. Luminex beads are available from Luminex Corporation, Austin, Tex., United States of America.

Preferably the assay used is a nephelometric or turbidimetric method. Nephelometric and turbidimetric assays for the detection of λ- or κ-FLC are generally known in the art, but not for total FLC assays. They have the best level of sensitivity for the assay. λ and κ FLC concentrations may be separately determined or a single assay for total FLC arrived at. Such an assay contains anti-κ and anti-λ FLC antibodies typically at a 60:40 ratio, but other ratios, such as 50:50 may be used.

Antibodies may also be raised against a mixture of free λ and free κ light chains.

The amount of total FLC may be compared to a standard, predetermined value to determine whether the total amount is higher or lower than a normal value. Patients with amounts above 50 mg/L FLC in serum, for example, has been shown to have significantly reduced survival.

Historically, assay kits have been produced for measurement of kappa and lambda FLC separately, to allow the calculation of a ratio. They have been conventionally used in individuals already exhibiting disease symptoms.

Preferably the assay is capable of determining FLC, for example total FLC, in the sample for example from approximately 1 mg/L to 100 mg/L, or 1 mg/L-80 mg/L. This is expected to detect the serum FLC concentrations in the vast majority of individuals without the requirement for re-assaying samples at a different dilution.

Preferably the method comprises detecting the amount of total free light chain in the sample utilising an immunoassay, for example, by utilising a mixture of anti-free κ light chain and anti-free λ light chain antibodies or fragments thereof. Such antibodies may be in a ratio of 50:50 anti-κ:anti-λ antibodies. Antibodies, or fragments, bound to FLC may be detected directly by using labelled antibodies or fragments, or indirectly using labelled antibodies against the anti-free λ or anti-free κ antibodies.

The antibodies may be polyclonal or monoclonal. Polyclonal may be used because they allow for some variability between light chains of the same type to be detected as they are raised against different parts of the same chain. The production of polyclonal antibodies is described, for example in WO97/17372.

Assay kits for FLC, for example for use in the methods of the invention are also provided. The kits may detect the total amount FLC in a sample. They may be provided in combination with instructions for use in the methods of the invention.

The assay kits may be adapted to detect an amount of total free light chain (FLC) in a sample below 25 mg/L, most preferably, below 20 mg/L or about, 10 mg/L, below 5 mg/L or 4 mg/L. The calibrator material typically measures the range 1-100 mg/L. The assay kit may be, for example, a nephelometric assay kit. Preferably the kit is an immunoassay kit comprising one or more antibodies against FLC. Typically the kit comprises a mixture of anti-κ and anti-λ FLC antibodies. Typically a mixture of 50:50 anti-free κ and anti-free λ antibodies are used. The kit may be adapted to detect an amount of 1-100 mg/L, or preferably 1-80 mg/L total free light chain in a sample.

Fragment of antibodies, such as (Fab)₂ or Fab antibodies, which are capable of binding FLC may also be used.

The antibodies or fragments may be labelled, for example with a label as described above. Labelled anti-immunoglobulin binding antibodies or fragments thereof may be provided to detect anti-free λ or anti-free κ bound to FLC.

The kit may comprise calibrator fluids to allow the assay to be calibrated at the ranges indicated. The calibrator fluids preferably contain predetermined concentrations of FLC, for example 100 mg/L to 1 mg/L, below 25 mg/L, below 20 mg/L, below 10 mg/L, below 5 mg/L or to 1 mg/L. The kit may also be adapted by optimising the amount of antibody and “blocking” protein coated onto the latex particles and, for example, by optimising concentrations of supplementary reagents such as polyethylene glycol (PEG) concentrations.

The kit may comprise, for example, a plurality of standard controls for the FLC. The standard controls may be used to validate a standard curve for the concentrations of the FLC or other components to be produced. Such standard controls confirm that the previously calibrated standard curves are valid for the reagents and conditions being used. They are typically used at substantially the same time as the assays of samples from subjects. The standards may comprise one or more standards below 20 mg/L for FLC, more preferably below 15 mg/L, below approximately 10 mg/L or below 5 mg/L, in order to allow the assay to detect the lower concentrations of free light chain.

The kit may also include one or more antibodies or other assays against one or more markers selected from GAM, CRP, bilirubin, cystatin C, creatinine, albumin, INR, AST and ALT preferably GAM, CRP, bilirubin, cystatin C, albumin and/or INR. Such antibodies and assays are generally known in the art and are available commercially.

The assay kit may be a nephelometric or turbidimetric kit. It may be an ELISA, flow cytometry, fluorescent, chemiluminescent or bead-type assay or dipstick. Such assays are generally known in the art.

The assay kit may also comprise instructions to be used in the method according to the invention. The instructions may comprise an indication of the concentration of total free light chain considered to be a normal value, below which, or indeed above which, shows an indication of either increased or decreased likelihood of the liver disease being present, for example. Such concentrations may be as defined above.

The invention will now be described by way of example only, with reference to the following figures:

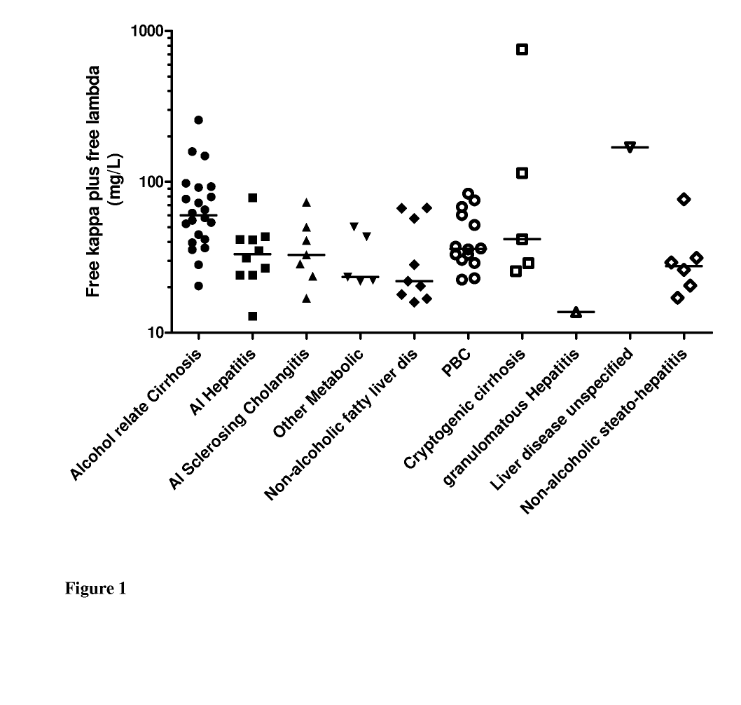

FIG. 1 shows the levels of total serum FLC concentrations in liver diseases. The median value is indicated by the black line.

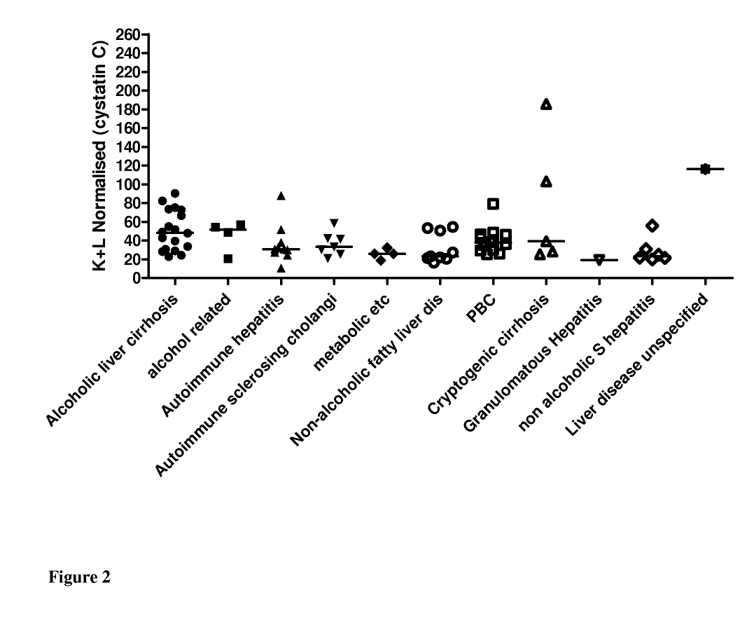

FIG. 2 shows FLC levels following correction for renal impairment in the liver diseases. The median value is indicated by the black line.

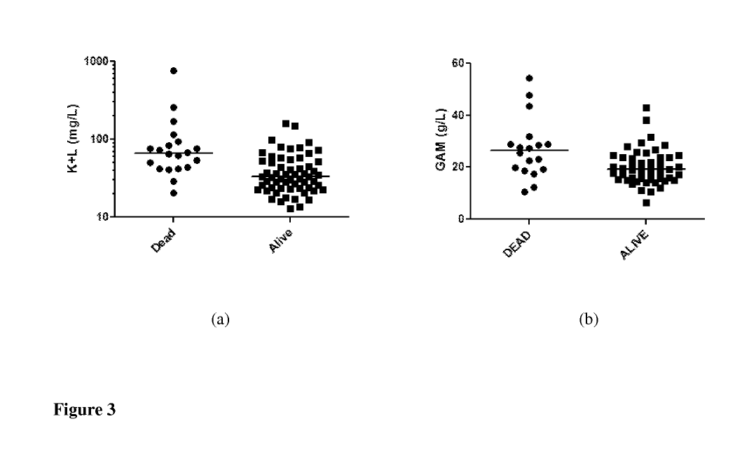

FIG. 3 shows the comparison of levels of total free light chains (kappa+lambda, mg/L) and total immunoglobulins (IgG, IgA and IgM) in patients who have died versus those known to be alive.

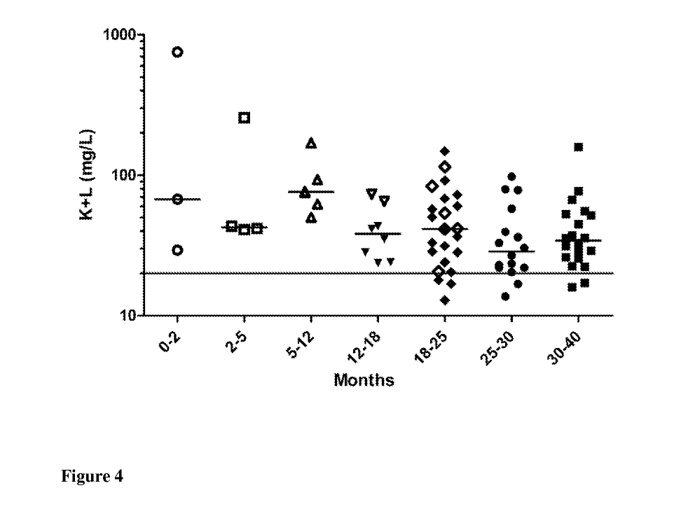

FIG. 4 shows total free light chain levels (kappa+lambda, mg/L) in patients with liver disease, segregated to highlight the deaths and patients who remain alive, since the sample was taken, until 200111. Open symbols indicate patients who have died and filled symbols indicate patients known to be alive. The median value is indicated by the black line.

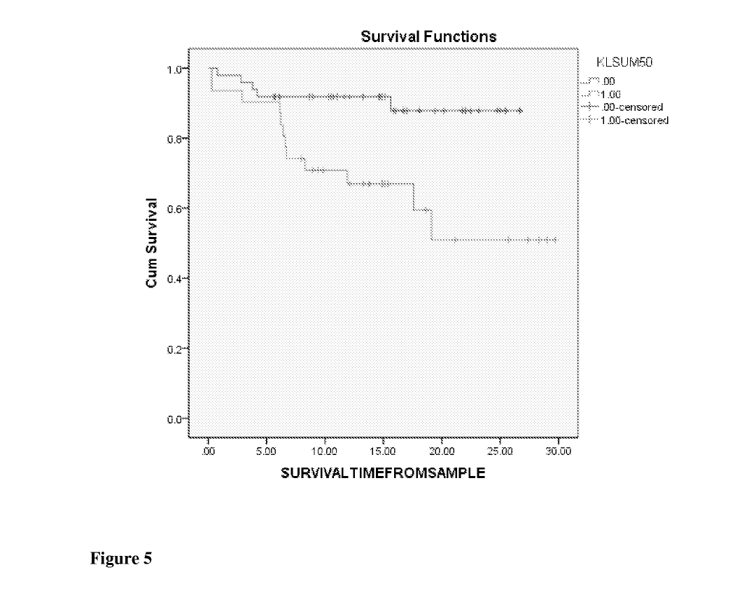

FIG. 5 shows Kaplan-Meier survival analysis of free light chain levels above and below 50 mg/L in liver disease, the survival time is shown in months.

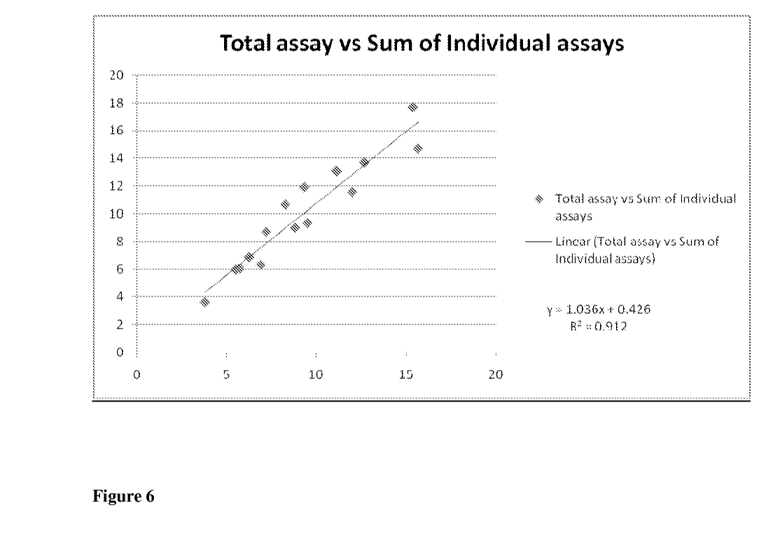

FIG. 6 is a comparison between the total FLC concentrations obtained using separate, commercially available, anti-free κ and anti-free λ assay kits, compared to a total FLC assay kit using combined anti-λ and anti-κ free light chain antibodies. Values are shown in mg/L with summated serum free light chains on the x-axis and total free light chains on the y-axis.

### LIVER DISEASE

### Methods

Serum samples from 80 patients with liver disease were obtained from the University Hospital, Birmingham, UK. The patients had a range of liver diseases including alcohol related liver disease, autoimmune hepatitis, autoimmune sclerosing cholangitis, non-alcoholic fatty liver disease, primary biliary cirrhosis (PBC), cryptogenic cirrhosis, granulomatous hepatitis, and non alcoholic steato-hepatitis.

The test assessments made included:

Serum FLC concentrations, both kappa and lambda (Freelite, The Binding Site, Birmingham, UK).

Total, serum FLC concentrations were calculated by adding the values for kappa FLC and lambda FLC. Values were compared against established normal ranges (κ: 3.3-19.4 mg/L, λ: 5.71-26.3 mg/L, ratio: 0.26-1.65).

Cystatin C as a measure of renal impairment (Cystatin C assay, The Binding Site, Birmingham, UK).

Cystatin C results were used to correct the FLC values for renal impairment. The FLC concentration was divided by the cystatin C value to give a correction for renal impairment. Immunoglobulins IgG, IgA & IgM (Standard tests in the art). Spearman Rank correlation analysis was used to examine associations between sFLCs and immunoglobulin concentrations.

### Results

79/80 samples had normal FLC ratios. However, sFLC concentrations were above the summated normal range in 39% of patients (FIG. 1). Data normalised for renal function indicated 34% of patients had elevated sFLCs independent of kidney impairment. Individuals in several groups had particularly high sFLC concentrations; highest levels were consistently detected in alcoholic LD (median concentration: 59.9 mg/L, range: 20.4-256.4 mg/L).

Corrected levels of FLCs in the liver diseases are shown in FIG. 2

Correlations were observed, although not in all cases between concentrations of sFLC and IgG (r=0.68, P<0.0001) and IgA (r=0.56, P<0.0001) although only weakly with IgM (r=0.28, P<0.0001). Correlation values given are calculated following correction of the FLC concentrations for renal impairment.

Further analysis of the survival versus levels of FLC and other markers of liver function, has been carried out (see for example FIG. 3). A comparison of various members of B-cell or liver function in liver disease patients who have died versus those known to be alive is shown in Table 1 below:

TABLE 1

| | Mann-Whitney |
|---|---|
| Variable | P value |
| K + L | 0.0001 |
| GAM | 0.01 |
| CRP | 0.007 |
| Bilirubin | 0.0012 |
| Cystatin C | 0.0001 |
| Creatinine | 0.15 NS |
| Albumin | <0.0001 |
| INR | 0.0004 |
| AST | 0.45 NS |
| ALT | 0.21 NS |
| PLATELET NUMBER | 0.02 |
| MELD score | 0.009 |

Abbreviations
K + L—total free kappa and free lambda
GAM—total immunoglobulins (IgA, IgG, IgM)
CRP—C-reactive protein
INR—International normalised ratio (a measure of blood clotting time)
AST—Aspartate Amino Transferase
ALT—Alanine Amino Transferase
MELD Score—Model for End Liver Disease Score

FIG. 4 shows patients who have died compared to those known to be alive.

FIG. 5 shows Kaplan-Meier survival analysis assessing FLC levels (>50 mg/L) in those who have died versus those who remain alive, showing increased survival at lower concentrations of FLC.

Cox regression analysis showed FLCs above 50 mg/L are a significant marker of death (hazard ratio 4.09, P=0.008).

### Discussion

Serum FLCs were polyclonally elevated in liver disease, particularly alcohol-related liver disease. This was only partly due to reduced clearance and mostly due to either increased production. In many, but not all patients, increased sFLC production was associated with raised immunoglobulin concentrations. On this basis the sFLCs are a sensitive marker of immune activation in liver disease and may be useful biomarkers for diagnosis and monitoring of inflammatory/immune-mediated liver disease patients.

### Assay Kit

The method according to the invention may utilise the following assay kit. The assay kit quantifies the total free κ plus free λ light chains present within patient samples, for example, in serum. This may be achieved by coating 100 nm carboxyl modified latex particles with a 50:50 blend of anti-free κ and anti-free λ light chain sheep antibody. In the assay exemplified below, the measuring range for the total free light chains is for 1-80 mg/L. However, other measuring ranges could equally be considered.

Anti-free κ and anti-free λ anti sera are produced using techniques generally known in the art, in this particular case in sheep. The general immunisation process is described in WO 97/17372.

Anti-κ and anti-λ antisera were diluted to equal concentrations using phosphate buffered saline (PBS). Those antibodies were combined to produce antisera comprising 50% anti lc antibody and 50% anti λ antibody.

Antibodies were coated onto carboxyl modified latex at a coat load of 10 mg/lot. This was achieved using standard procedures. See, for example, “Microparticle Reagent Optimization: A laboratory reference manual from the authority on microparticles” Eds: Caryl Griffin, Jim Sutor, Bruce Shull. Copyright Seradyn Inc, 1994 (P/N 0347835(1294).

This reference also provides details of optimising the assay kits using polyethylene glycol (PEG).

The combined antibodies were compared to results obtained using commercially available lc and λ Freelite™ kits (obtained from the Binding Site Group Limited, Birmingham, United Kingdom). Such Freelite™ kits identify the amount of κ and the amount of λ free light chains in separate assays. The total FLC kits were used to generate curves, which were validated using controlled concentrations. Calibration curves were able to be obtained between 1 and 80 mg/l for total free light chain. In the results table below, results were obtained for κ free light chain (KFLC), λ free light chain (LFLC) and total FLC, using the lc Freelite™, λ Freelite™ and total free light chain assays. These results are shown for 15 different normal serum samples. The results are shown in the table below and in FIG. 2 as measured by turbidimetry.

Preliminary results indicate that the principle of using a total free light chain assay based on anti-κ and anti-λ free light chain antibody is viable.

### Results

| | | | | | | % diff Total FLC |
|---|---|---|---|---|---|---|
| | Batch | Results (mg/l) | | | KFLC + | vs (KFLC + |
| USN | Id | KFLC | LFLC | Total FLC | LFLC | LFLC) |
| 1 | 104 | 3.37 | 3.51 | 6.31 | 6.88 | −8.3% |
| 2 | 151 | 3.42 | 5.39 | 8.99 | 8.81 | 2.0% |
| 3 | 158 | 3.28 | 6.21 | 9.35 | 9.49 | −1.5% |
| 4 | 161 | 2.05 | 3.62 | 6.06 | 5.67 | 6.9% |
| 5 | 179 | 6.83 | 5.84 | 13.71 | 12.67 | 8.2% |
| 6 | 180 | 2.19 | 3.27 | 5.96 | 5.46 | 9.2% |
| 7 | 181 | 2.98 | 5.27 | 10.64 | 8.25 | 29.0% |
| 8 | 182 | 4.72 | 7.26 | 11.6 | 11.98 | −3.2% |
| 9 | 216 | 2.54 | 4.66 | 8.7 | 7.2 | 20.8% |
| 10 | 217 | 3.01 | 3.24 | 6.88 | 6.25 | 10.1% |
| 11 | 219 | 7.12 | 8.53 | 14.73 | 15.65 | −5.9% |
| 12 | 227 | 1.47 | 2.31 | 3.66 | 3.78 | −3.2% |
| 13 | 228 | 8.16 | 7.2 | 17.67 | 15.36 | 15.0% |
| 14 | 229 | 4.51 | 6.61 | 13.1 | 11.12 | 17.8% |
| 15 | 231 | 3.69 | 5.6 | 11.91 | 9.29 | 28.2% |
| | | | | | Mean | 8.3% |
| | | | | | Diff | |

What is claimed is:

1. A method of identifying a subject likely to have liver disease, or for determining the prognosis of a subject previously identified as having a liver disease comprising detecting an amount of free light chains in a sample from the subject, wherein a higher amount of FLC is associated with an increased likelihood of the subject having a liver disease or an increased likelihood of having a poor prognosis of a liver disease.

2. A method according to claim 1 for following the treatment of a liver disease in the subject, wherein a lower level of FLC after treatment indicates that the treatment is working.

3. A method according to claim 2, wherein the detected amount of free light chains is the total free light chains present in the sample.

4. A method according to claim 1, wherein the FLC is determined in a sample of serum from the subject.

5. A method according to claim 1, wherein the total FLC is determined by immunoassay using anti-free light chain antibodies.

6. A method according to claim 5, wherein the antibodies are a mixture of anti-free κ light chain and anti-free λ light chain antibodies.

7. A method according to claim 1, wherein the method comprises detecting the amount of FLC by nephelometry or turbidimetry.

8. A method according to claim 1, wherein the subject does not have symptoms of B-cell associated disease.

9. An assay kit for use in the method according to claim 1, said kit comprising
one or more anti-FLC antibodies; and
a normal value against which a concentration of FLC obtained using the assay kit, indicates an increased risk of liver disease in a subject; and
instructions to be used in the method.

10. (canceled)

11. An assay kit according to claim 9, further comprising one or more antibodies against one or more markers selected from GAM, CRP, bilirubin, CystatinC, creatinine, albumin, INR, AST and ALT.

12. A method of prognosing a subject at risk of loss of liver function, said method comprising
analyzing a sample isolated from said subject to measure the amount of free light chains (FLC) present in said sample;
determining if the detected concentration of free light chains (FLC) present in said sample exceeds a normal value, wherein an amount of FLC higher than the normal value is associated with an increased risk of loss of liver function in said subject.
